(19) United States
(12) Patent Application Publication
(10) Pub. No.: US 2011/0143415 A1
(43) Pub. Date: Jun. 16, 2011

(54) REPROGRAMMING NORMAL AND CANCEROUS HUMAN CELL LINES INTO HUMAN INDUCED POLURIPOTENT STEM CELLS BY CO-ELECTROPORATION WITH LIVING XENOPUS LAEVIS FROG OOCYTES

(76) Inventor: Sergei Paylian, Wisconsin Dells, WI (US)

(21) Appl. No.: 12/960,527

(22) Filed: Dec. 5, 2010

Related U.S. Application Data
(60) Provisional application No. 61/286,241, filed on Dec. 14, 2009.

(51) Int. Cl. C12N 13/00; C12N 5/071

(52) U.S. Cl. 435/173.6; 435/377; 435/325

(57) ABSTRACT

Using electroporation, it is possible to activate the natural reprogramming potential of living Xenopus laevis oocytes and pass it on to donor cells placed with eggs in one electroporation chamber. We demonstrated that co-electroporation at 150 v/cm/25 μF of mature oocytes with ^{˜}10⁵ cells/ml of suspension of various normal and cancerous human cell lines, such as bone marrow stromal cells, foreskin fibroblasts, pre-adipocytes, CD4+ T-lymphocytes, cheek cells, cervical carcinoma (HeLa) cells and breast adenocarcinoma (MCF-7) cells, reprograms donor cells into iPSc-like cells, which form colonies on irradiated MEF feeders. The iPSc-like cells generated by this study resemble human embryonic stem cells in colony morphology and expression of stem cell-associated transcription factors, including Oct3/4, Nanog, SOX-2, Rex-1, TRA-1-60 and SSEA-1. New method obviates the use of retroviral or lentiviral gene delivery vectors and other “non-parental” reprogramming approaches.

I claim priority to my earlier filed provisional patent application Ser. No. 61/286,241 filed Dec. 14, 2009

### FIELD OF THE INVENTION

The present invention relates to stem cells. More specifically, the present invention relates to methods of generating pluripotent stem cells from differentiated cells.

### BACKGROUND

“Regression” of specialized cells or tissues to a simpler, embryonic-like, unspecialized form, otherwise known as “dedifferentiation,” is a widespread event in the living world. This phenomenon is observed at almost every level of organismal complexity. It is present, for example, in bacteria, the soil-living amoeba Dictyostelium discoideum (1), plants such as tobacco (Nicotiana tabacum) (2), and animals such as red-spotted newts (Notophthalmus viridescens) and axolotls (Ambystoma punctatum) (3), etc. It is important that de-differentiated cells keep an epigenetic memory of their tissue of origin, which ensures their successful re-differentiation back into damaged cells; axolotl cells exhibit this requirement during limb regeneration (Kragl et al., 2009). Recently, Kim K. et al. showed that epigenetic memory is also present in induced pluripotent stem cells (Kim K. et al, 2010). All of the results noted above indicate that a universal mechanism for reprogramming may exist in nature and that any divergence from the RP tools observed in nature may result in the failure or inconsistency of our efforts to reprogram cells. Such recalcitrance, for example, may occur during the preparation of crude extracts from Xenopus oocytes, in which vital nucleocytoplasmic communications present in living eggs are completely disrupted. In other words, the reprogramming machinery of frog oocytes, if left intact, may work more effectively than other RP strategies.

In humans, stem cells uniquely have the ability to differentiate. Scientific teams working in the stem cell research field are moving in five promising directions to elucidate the biochemical properties and potential therapeutic usage of human embryonic stem cells. A first direction involves the identification of molecular mechanisms that play a key role in restricting pluripotency of embryonic stem cells. The endeavor may facilitate an understanding of why pluripotency is lost in adult cells, as well as when, how, and why a stem cell differentiates into another type of cell. A second direction involves the identification of sources of adult stem cells. Adult stem cells possess the ability to trans-dedifferentiate into virtually any of 210 known distinct human cell types. A third direction involves identification of reprogramming factors. Under specific conditions, reprogramming factors can trigger dedifferentiation of adult somatic cells into viable induced pluripotent stem (iPS) cells able to proliferate in an undifferentiated state while retaining pluripotency. A fourth direction includes nanotechnological approaches of studying formation of human tissue. This involves differentiation of embryonic stem cells in three-dimensional culture using different types of scaffolds and in conditions closely mimicking biochemical and physiological cellular interactions. Finally, a fifth direction includes moving experimental animal work into clinical trials.

As a result of this research, four key pluripotency genes involved in the production of pluripotent stem cells have been determined. These include: Oct-3/4, SOX2, c-Myc, and Klf4. These genes, in combination with other biochemical substances and chemicals, constitute the major focus in current somatic cell-stem cell reprogramming studies. However, one of these genes, c-Myc, is oncogenic. Twenty percent of chimeric mice expressing this gene develop cancer. Methods of generating iPS cells using transcription factors other than c-Myc have been reported. These methods do not appear to promote cancer but take much longer and are not as efficient.

In addition to tumorigenicity, another problem plaguing reprogramming methods is the low efficacy in reprogramming donor cells into iPS cells. According to various sources, RP efficacy could be as low as 0.5% with standard, four-factor retroviral RP (Meissner et al., 2007; Condic and Rao, 2008); 0.98%-2.34% when adding two more reprogramming factors (Markoulaki et al., 2009); 2%-4% with the use of dox-inducible lentiviruses (Wernig et al., 2008); and 18% with cell-to-cell extracts (Håkelien et al., 2002). Recently, new, non-viral approaches for improving RP efficacy were reported. These methods include the use of recombinant proteins (Zhou et al., 2009); the use of DHP-derivative (novel anti-oxidant) and low oxygen-tension conditions (Jee et al., 2010); the application of embryonic stem cell-specific microRNAs (Judson et al. , 2009); zinc-finger nucleases (Hockemeyer et al., 2009); drugs (Markoulaki et al., 2009; Huangfu et al., 2008; Wernig et al., 2008); hypoxia (Yoshida et al., 2009); silencing of the p53 pathway, which prevents mutations and preserves the genomic sequence (Hong et al., 2009); and ES cell-derived protein extracts (Cho et al., 2010). We believe that the low efficacy of reprogramming may be explained by the fact that the RP tools currently used in studies differ considerably from the delicate reprogramming machinery naturally present in living organisms. Nevertheless, these studies revealed some interesting events that preclude the de-differentiation of donor cells into the progenitor stage: active remodeling of somatic nuclei by the nucleosomal ATPase ISWI (Kikyo, 2000); reversible disassembly of somatic nucleoli by the germ cell proteins FRGY2a and FRGY2b (Gonda, 2003); the role of BRG1 and nucleoplasmin in chromatin decondensation and nuclear reprogramming (Hansis et al., 2004; Tamada et al., 2007); and the role of histone H3 lysine 4 methylation in the transcriptional reprogramming efficiency of somatic nuclei (Murata et al., 2010). Reprogramming events observed in mammalian somatic cells induced by Xenopus laevis egg extracts were described by Miyamoto and colleagues (Miyamoto et al., 2007). Other studies have shown that reprogramming methods have been able to induce iPS cells to express genes previously expressed only by embryonic stem cells. For example, human iPS cells express markers specific to human embryonic stem cells (hESCs). These markers include SSEA-3, SSEA-4, TRA-1-60, TRA-1-81, TRA-2-49/6E, and Nanog. Likewise, mouse iPS cells express SSEA-1 (but not SSEA-3 or SSEA-4) in a manner similar to mouse embryonic stem cells (mESCs). Reprogramming methods have also been able to induce activation of crucial transcriptional regulators putatively required for the reprogramming of somatic cells. These include Oct-3/4 and certain members of the Sox gene family (Sox1, Sox2, Sox3, and Sox15). Additional genes, including Klf1, Klf2, Klf4, Klf5, C-myc, L-myc, and N-myc, Nanog, and LIN28, have been identified as increasing induction efficiency.

Current methods of inducing dedifferentiation, however, confront many problems. First, despite many advances, available data indicates that the efficacy of contemporary methods of reprogramming of human somatic cells into iPS cells is still inadequate for suitable use in regenerative medicine. Second, the “forced” expression of certain genes and other manipulations used in some methods may cause unpredictable changes in the genetic makeup of the dedifferentiated cells. In addition, such approaches employ only a few of the many factors involved in the reprogramming process and thereby disrupt the integrity of the reprogramming machinery as a whole.

There is a need for an integrated, natural, efficacious, safe, consistent, and reversible method of reprogramming somatic cells for use in regenerative medicine.

Present invention concerns novel technique which allows the utilization of the natural reprogramming potential of living Xenopus laevis oocytes. New approach enables to generate human iPS cells in a consistent, safe and controllable way, creating a system which:
1. is fast, efficient, and free of ethical controversy
2. conducts human somatic cell de-differentiation in a highly reproducible, standardized fashion when applied to different cell lines
3. is able to effect “real-time” reprogramming
4. segregates human and amphibian components and simultaneously activates mutual semiochemical interactions
5. makes the somatic-cell reprogramming process controllable
6. is able to produce partially reprogrammed cells, which may represent the correct transitional point for successful re-differentiation or trans-differentiation reprogramming.

New invention describes experimental protocol which utilizes the electroporation of living Xenopus laevis oocytes as a powerful RP tool through co-electroporating living Xenopus laevis oocytes with human donor cells in one electroporation chamber. We refer to this new procedure as “BQ-activation.” The electropermeabilization ability of Xenopus laevis oocytes was investigated in earlier DNA transfection studies (Falk et al., 2007), and the RP effectiveness of electroporation was examined in studies on newts (Atkinsona et al., 2006), in which in vivo cellular electroporation induced de-differentiation in intact newt limbs. Relevant data for establishing appropriate electroporation parameters for frog oocytes came from published material on the electroporation of zebra fish eggs (Buono and Linser, 1992), Japanese killifish embryos (Hostetler et al., 2003) and from experiments on the electroporation of adipocytes within mouse adipose tissue (Granneman et al., 2004). In our studies, we demonstrated that co-electroporation, with pulses of 150 v/cm/25 nF/7 pulses, of living Xenopus laevis oocytes with different normal and cancerous human cells lines, such as human bone marrow stromal cells (BMSC), human foreskin fibroblasts (BJ cells), human pre-adipocytes (HPA cells), human peripheral blood CD4⁺ T-lymphocytes, human buccal (cheek) cells, human cervical carcinoma (HeLa) cells and human breast adenocarcinoma (MCF-7) cells, reprograms donor cells into iPSc-like cells, which form colonies on irradiated MEF (iMEF) feeders. The iPSc-like cells produced with this protocol resemble human embryonic stem cells in colony morphology and the expression of stem-cell associated transcription factors Oct3/4, Nanog, SOX-2, Rex-1, TRA-1-60, SSEA-1, and SSEA4 and the efficacy of reprogramming (calculated only for CD4+lymphocytes) was 23.4±3.5%. Importantly, this new method obviates the use of retroviral or lentiviral gene-delivery vectors and other “non-parental” reprogramming approaches and may hold great promise as a means of rapid and inexpensive production of human autologous stem cells.

### SUMMARY OF THE INVENTION

The present invention concerns methods for non-viral induction of human pluripotent stem cells derived from non-pluripotent, differentiated donor cells. The methods employ a natural reprogramming machinery such as living Xenopus laevis frog oocytes and electrical forces as appropriate reprogramming tools. The non-pluripotent cells that can be used in the present invention include but are not limited to human bone marrow stromal cells (BMSC), human foreskin fibroblasts (BJ cells), human pre-adipocytes (HPA cells), human peripheral blood CD4⁺ T-lymphocytes, human buccal (cheek) cells, human cervical carcinoma (HeLa) cells and human breast adenocarcinoma (MCF-7) cells. These cells are preferably derived from humans. In a preferred version, the method of inducing pluripotent stem cells involves their co-electroporation with living Xenopus laevis frog oocytes. In another version, the donor cells may be also be electroporated without the presence of frog oocytes. In yet another version, the donor cells may be cultured with frog oocytes without being electroporated.

One version of the present invention is a method of generating an induced pluripotent stem cell from a differentiated cell comprising co-electroporating the differentiated cell with a live oocyte.

Another version of the present invention is a method of generating an induced pluripotent stem cell from a differentiated cell comprising electroporating the differentiated cell.

An additional version of the present invention is a method of generating an induced pluripotent stem cell from a differentiated cell comprising co-incubating the differentiated cell with a live oocyte.

In any of the versions of the methods described herein, the method may induce expression of Oct-3/4, NANOG, SOX2, TRA-1-60 and/or Rex-1 in the cell. Additionally, in methods including electroporation, the electroporation step may comprise stimulating the differentiated cell with seven 50-volt/25 nF impulses with 1-second intervals and with a time constant equal to 0.5-0.7 milliseconds.

The invention is also directed to an induced pluripotent stem cell line generated by any of the methods described herein.

The methods of cellular reprogramming described herein confer several advantages. The reprogramming methods described herein lead to more rapid reprogramming than prior methods. Specifically, distinguished clusters of iPS cells successfully proliferating on supporting mouse embryonic fibroblast feeder cells can be observed in only 3 to 5 days using the methods described herein, compared to the 14-21 days previously reported.

In addition, the reprogramming methods described herein are much more efficient than prior methods. Reprogramming efficacy using the methods of the present invention can reach 23.4±3.5% which is more than 20 times higher than the efficacy presently yielded using prior methods.

The methods described herein are capable of inducing dedifferentiation in easily obtained human somatic cells, such as human bone marrow stromal cells (BMSC), human foreskin fibroblasts (BJ cells), human pre-adipocytes (HPA cells), human peripheral blood CD4⁺ T-lymphocytes, human buccal (cheek) cells, human cervical carcinoma (HeLa) cells and human breast adenocarcinoma (MCF-7) cells. The iPS cell lines derived from the methods described herein are non-immunogenic.

Finally, the reprogramming methods described herein obviate the use of retroviral and lentiviral gene delivery vectors and other interventions that may cause unpredictable and irreversible changes in the genetic makeup of donor cells. The iPS cells derived from the present methods are indistinguishable from human embryonic stem cells in colony morphology, growth properties, and expression of pluripotency-associated transcription factors.

The objects and advantages of the invention will appear more fully from the following detailed description of the preferred embodiment of the invention made in conjunction with the accompanying images.

### BRIEF DESCRIPTION OF THE DRAWINGS

FIG. 1 shows de-differentiation of human BMSCs Into human iPSc-like cells (7 days after BQ-activation, 20×).

FIG. 2. shows de-differentiation BMSCGFP Into human iPSc-like cells ((7 days after BQ-activation, 40×).

FIG. 3 shows BQ-activated BJ-iPSc-like cells at different days post-activation: AP staining, 5 days; Oct 3/4, 5 Days; Nanog, 5 days; TRA-1-60, 9 day; Rex-1, Sox-2 , 5 days.

FIG. 4 de-differentiation of HPA cells Into human iPSc-like cells. (A): HPA control. B-random area. (C-F): Different stages of reprogramming of HPA-iPSc-like cells, where (C) is area of starting point of observations of the same cluster shown on figures C, D, E, and F. (F-G): phase contrast images of the same cluster which by 5th day after BQ-activation is now completely formed (H): AP staining (I-J): Oct 3/4. (K- L): Nanog, (M-N): Sox-2. (O-P): TRA-1-60. (Q-R): Rex-1.

FIG. 5 shows light microscope images of subcultured primary HPA-iPSc-like cells at different stages of cluster formation. (A): random area. (B, C, D): same area.

FIG. 6 shows de-differentiation of CD4+ T-Lymphocytes Into human iPSc-like cells. (A): human CD4TL, control, 20×, (B): CD4TL co-cultured with iMEF feeder cells, no activation, 20×. (C-D): CD4TL-iPSc,-like cluster 5 days, 10×-20×. (E-F): Lower part of cluster D, 20×-40×, 5 days. (G-H): AP-staining, 9 days, 20×-40×. (I-J): Two adjoin clusters of CD4TL-iPS cells, Oct 3/ 4, 10 days, 20×. (K-L): Nanog. 10 days, 10×. (M): Sox-2, 5 days, 20×. (N): CD4+TL-iPSc like cluster, Rex-1, 5 days, 20×. (O): DAPI staining corresponding to Sox-2 and Rex-1 staining (P): Colors combined. (Q-R): SOX-2, (10 days), 40×. (S-T): TRA-1-60 (9 days), 40×. (U-V): Rex-1 (10 days), 20×.

FIG. 7 shows de-differentiation of human cheek cells into human iPSc-like cells. (A, B, C, D): Different stages of formation of BU-iPSc-like clusters. A—clearly distinguished cheek cells attached to iMEF feeders; B—formation of multiple clusters of BU-iPSc-like cells, which are surrounded with non-differentiated buccal cells; C—single BU-iPSc-like cluster; D—the same cluster; E—BU-iPSc-like cluster with possible signs of differentiation (data not analyzed). F—the same cluster at a higher magnification; G—large cluster of subcultured Bu-iPSc on iMEF feeders; H—cluster of Bu-iPSc growing in a feeder free environment on StemAdhere™ substrate.

FIG. 8 shows expression of human ES markers by BU-iPSc-like cells. (A-B): Oct 3/4 gene, 96 h, 20×. (C-D): Nanog, 10 days, 20×. (E-F): Sox-2, 10 days, 40×. (G-H): TRA-1-60, 9 days, 40×. (I-J): Rex-1, 11 days, 40×.

FIG. 9 shows de-differentiation of human cervical carcinoma (HeLa) cells Into human iPSc-like cells. (A): HeLa cells control, 20×. (B): HeLa cells cultured on iMEF feeders (no BQ-activation), 20×. (C): HeLa-iPSc-like cluster, 5 days, 10×. (D): Another HeLa-iPSc-like cluster, 8 days, 10×. (E-F): Expression of Oct 3/4 gene in fully de-differentiated cluster of HeLa-iPSc-like cells, 11 days, 20×. (G-H): Oct 3/4 expression in partially de-differentiated cluster of HeLa-iPSc-like cells, 11 days, 20×.

FIG. 10 shows de-differentiation of human breast adenocarcinoma (MCF-7) cells Into human iPSc-like cells (A): MCF-7 control, 10×. (B): MCF-7 cells cultured on iMEF feeders (no BQ-activation), 40×. (C-D): Expression of Oct 3/4 genes in fully de-differentiated cluster of MCF-7 iPSc-like cells, 11 days, 40×. (E-F): Nanog gene expression in partially de-differentiated cluster of MCF-7-iPSfc-like cells, 11 days, 40×.

FIG. 11 shows early expression of Nanog gene in human CD4+ lymphocytes used for the calculation of the efficacy of reprogramming of hCD4TL. (A): 20× image of human hCD4TL in the field of phase contrast microscope, 24 h after BQ-activation. (B): Same area in the field of fluorescent microscope (lymphocytes strongly express Nanog gene [symbol] ).

### DETAILED DESCRIPTION OF THE INVENTION

Abbreviations: The following abbreviations are used herein:
AP=alkaline phosphatase;
BJ cells=a type of human foreskin fibroblast cells;
BMSC=bone marrow stromal cells
CD4⁺T=peripheral blood CD4-positive T-lymphocytes;
GFP=green fluorescent protein
hESC=human embryonic stem cells;
HeLa=human cervical carcinoma cells;
HPA=human pre-adipocytes;
iMEF=mouse embryonic fibroblasts;
iPS cells=induced pluripotent stem cells;
MCF-7=breast adenocarcinoma cells.

Differentiated Cells: The present invention is directed to methods of generating induced pluripotent cells from differentiated cells. Cells such as human bone marrow stromal cels, foreskin fibroblasts, pre-adipocytes, peripheral blood CD4⁺ T-lymphocytes, buccal mucosa cells, and cancer cells such as HeLa cervical carcinoma cells and MCF-7 breast carcinoma cells are preferred. However, it is envisioned that any differentiated cell may be used.

For example, the present method may be used with cells of the integumentary system. These cells include keratinizing epithelial cells such as epidermal keratinocytes (differentiating epidermal cells), epidermal basal cells (stem cells), keratinocytes of fingernails and toenails, nail bed basal cells (stem cells), medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, and hair matrix cells (stem cells).

Other cells of the integumentary system include wet stratified barrier epithelial cells such as surface epithelial cells of stratified squamous epithelium of cornea, tongue, oral cavity, esophagus, anal canal, distal urethra and vagina, basal cells (stem cells) of epithelia of cornea, tongue, oral cavity, esophagus, anal canal, distal urethra and vagina, and urinary epithelium cells (lining urinary bladder and urinary ducts).

The present method can also be used with exocrine secretory epithelial cells. These cells include salivary gland mucous cells (polysaccharide-rich secretion), salivary gland serous cells (glycoprotein enzyme-rich secretion), Von Ebner's gland cells in tongue (washes taste buds), mammary gland cells (milk secretion), lacrimal gland cells (tear secretion), ceruminous gland cells in ear (wax secretion), eccrine sweat gland dark cells (glycoprotein secretion), eccrine sweat gland clear cells (small molecule secretion), apocrine sweat gland cells (odoriferous secretion, sex-hormone sensitive), Gland of Moll cells in eyelid (specialized sweat gland), sebaceous gland cells (lipid-rich sebum secretion), Bowman's gland cells in nose (washes olfactory epithelium), Brunner's gland cells in duodenum (enzymes and alkaline mucus), seminal vesicle cells (secretes seminal fluid components, including fructose for swimming sperm), prostate gland cells (secretes seminal fluid components), bulbourethral gland cells (mucus secretion), Bartholin's gland cells (vaginal lubricant secretion), Gland of Littre cells (mucus secretion), uterus endometrium cells (carbohydrate secretion), isolated goblet cells of respiratory and digestive tracts (mucus secretion), stomach lining mucous cells (mucus secretion), gastric gland zymogenic cells (pepsinogen secretion), gastric gland oxyntic cells (hydrochloric acid secretion), pancreatic acinar cells (bicarbonate and digestive enzyme secretion), Paneth cells of small intestine (lysozyme secretion), Type II pneumocytes of lung (surfactant secretion), and Clara cells of lung.

The present method can also be used with hormone secreting cells. These include anterior pituitary cells such as somatotropes, lactotropes, thyrotropes, gonadotropes, and corticotropes. Other hormone secreting cells include intermediate pituitary cells, magnocellular neurosecretory cells, gut and respiratory tract cells, thyroid gland cells (thyroid epithelial cells and parafollicular cells), parathyroid gland cells (parathyroid chief cells and oxyphil cells), adrenal gland cells (chromaffin cells), Leydig cells of testes, theca interna cells of ovarian follicle, corpus luteum cells of ruptured ovarian follicle (including granulosa lutein cells and theca lutein cells), Juxtaglomerular cells (renin secretion), macula densa cells of kidney, peripolar cells of kidney, and mesangial cells of kidney.

The present method can also be used with metabolism and storage cells. These cells include hepatocytes (liver cells), white fat cells, brown fat cells, and liver lipocytes.

The present method can also be used with barrier function cells such as those found in the lung, gut, exocrine glands and urogenital tract. Other barrier function cells may include those of the kidney such as kidney glomerulus parietal cells, kidney glomerulus podocytes, kidney proximal tubule brush border cells, Loop of Henle thin segment cells, kidney distal tubule cells, and kidney collecting duct cells. Yet other barrier functions cells may include type I pneumocytes (lining air space of lung), pancreatic duct cells (centroacinar cells), nonstriated duct cells (of sweat gland, salivary gland, mammary gland, etc.), duct cells (of seminal vesicle, prostate gland, etc.), intestinal brush border cells (with microvilli), exocrine gland striated duct cells, gall bladder epithelial cells, ductulus efferens nonciliated cells, epididymal principal cells, and epididymal basal cells.

The present invention can also be used with epithelial cells lining closed internal body cavities. These include blood vessel and lymphatic vascular endothelial fenestrated cells, blood vessel and lymphatic vascular endothelial continuous cells, blood vessel and lymphatic vascular endothelial splenic cells, synovial cells (lining joint cavities, hyaluronic acid secretion), serosal cells (lining peritoneal, pleural, and pericardial cavities), squamous cells (lining perilymphatic space of ear), squamous cells (lining endolymphatic space of ear), columnar cells of endolymphatic sac with microvilli (lining endolymphatic space of ear), columnar cells of endolymphatic sac without microvilli (lining endolymphatic space of ear), dark cells (lining endolymphatic space of ear), vestibular membrane cells (lining endolymphatic space of ear), stria vascularis basal cells (lining endolymphatic space of ear), stria vascularis marginal cells (lining endolymphatic space of ear), Cells of Claudius (lining endolymphatic space of ear), Cells of Boettcher (lining endolymphatic space of ear), choroid plexus cell (cerebrospinal fluid secretion), pia-arachnoid squamous cells, pigmented ciliary epithelium cells of eye, nonpigmented ciliary epithelium cells of eye, and corneal endothelial cells.

The present invention may also be used with ciliate cells with propulsive function such as respiratory tract ciliated cells, oviduct ciliated cells (in female), uterine endometrial ciliated cells (in female), rete testis ciliated cells (in male), ductulus efferens ciliated cells (in male), and ciliated ependymal cells of central nervous system (lining brain cavities).

The present invention may also be used with extracellular matrix secretion cells such as ameloblast epithelial cells (tooth enamel secretion), planum semilunatum epithelial cells of vestibular apparatus of ear (proteoglycan secretion), organ of Corti interdental epithelial cells (secreting tectorial membrane covering hair cells), loose connective tissue fibroblasts, corneal fibroblasts (corneal keratocytes), tendon fibroblasts, bone marrow reticular tissue fibroblasts, other nonepithelial fibroblasts, pericytes, nucleus pulposus cells of intervertebral discs, cementoblasts/cementocytes (tooth root bonelike cementum secretion), odontoblasts/odontocytes (tooth dentin secretion), hyaline cartilage chondrocytes, fibrocartilage chondrocytes, elastic cartilage chondrocytes, osteoblasts/osteocytes, osteoprogenitor cells (stem cells of osteoblasts), hyalocytes of vitreous body of eye, stellate cells of perilymphatic space of ear, hepatic stellate cells (Ito cells), and pancreatic stellate cells.

The present invention may also be used with contractile cells such as skeletal muscle cells. Skeletal muscle cells include red skeletal muscle cells (slow), white skeletal muscle cells (fast), intermediate skeletal muscle cells, nuclear bag cells of muscle spindle, and nuclear chain cells of muscle spindle. Other contractile cells include satellite cells (stem cells), heart muscle cells (ordinary heart muscle cells, nodal heart muscle cells, and Purkinje fiber cells), smooth muscle cells (various types), myoepithelial cells of iris, and myoepithelial cells of exocrine glands.

The present invention may also be used with blood and immune system cells such as erythrocytes (red blood cells), megakaryocytes (platelet precursor), monocytes, connective tissue macrophages (various types), epidermal Langerhans cells, osteoclasts (in bone), dendritic cells (in lymphoid tissues), microglial cells (in central nervous system), neutrophil granulocytes, eosinophil granulocytes, basophil granulocytes, mast cells, helper T cells, suppressor T cells, cytotoxic T cells, Natural Killer T cells, B cells, natural killer cells, reticulocytes, and stem cells and committed progenitors for the blood and immune system (various types).

The present invention may also be used with cells of the nervous system. These cells include sensory transducer cells such as auditory inner hair cells of organ of Corti, auditory outer hair cells of organ of Corti, basal cells of olfactory epithelium (stem cells for olfactory neurons), gold-sensitive primary sensory neurons, heat-sensitive primary sensory neurons, Merkel cells of epidermis (touch sensor), olfactory receptor neurons, pain-sensitive primary sensory neurons (various types), photoreceptor cells of retina in eye (photoreceptor rod cells, photoreceptor blue-sensitive cone cells of eye, photoreceptor green-sensitive cone cells of eye, and photoreceptor red-sensitive cone cells of eye), proprioceptive primary sensory neurons (various types), touch-sensitive primary sensory neurons (various types), Type I carotid body cells (blood pH sensor), Type II carotid body cells (blood pH sensor), Type I hair cells of vestibular apparatus of ear (acceleration and gravity), Type II hair cells of vestibular apparatus of ear (acceleration and gravity), and Type I taste bud cells. Other nervous system cells may include autonomic neuron cells such as cholinergic neural cells (various types), adrenergic neural cells (various types), and peptidergic neural cells (various types).

Other nervous system cells may include sense organ and peripheral neuron supporting cells such as inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, Type I taste bud supporting cells, olfactory epithelium supporting cells, Schwann cells, satellite cells (encapsulating peripheral nerve cell bodies), and enteric glial cells.

Other nervous system cells may include central nervous system neurons and glial cells such as astrocytes (various types), neuron cells (large variety of types, still poorly classified), oligodendrocytes, and spindle neurons.

Yet other nervous systems cells may include lens cells such as anterior lens epithelial cells, and crystallin-containing lens fiber cells.

The present invention may also be used with pigment cells such as melanocytes and retinal pigmented epithelial cells.

The present invention may also be used with germ cells such as oogonia/oocytes, spermatids, spermatocytes, spermatogonium cells (stem cells for spermatocytes), and spermatoza.

The present invention may also be used with nurse cells such as ovarian follicle cells, Sertoli cells (in testis), and thymus epithelial cells.

The present invention may also be used with interstitial cells such as interstitial kidney cells.

The present invention may also be used with any cancer cell. Exemplary cancer cells include those derived from the following types of cancer: Adult acute lymphoblastic leukemia, childhood acute lymphoblastic leukemia, adult acute myeloid leukemia, childhood acute myeloid leukemia, adrenocortical carcinoma, childhood adrenocortical carcinoma, AIDS-related cancers, AIDS-related lymphoma, anal cancer, appendix cancer, childhood astrocytomas, childhood atypical teratoid/rhabdoid tumor (central nervous system), basal cell carcinoma, extrahepatic bile duct cancer, bladder cancer, childhood bladder cancer, bone cancer (osteosarcoma and malignant fibrous histiocytoma), childhood brain stem glioma, adult brain tumor, childhood brain tumor (brain stem glioma), childhood brain tumor (central nervous system atypical teratoid/rhabdoid tumor), childhood brain tumor (central nervous system embryonal tumors), childhood brain tumor (astrocytomas), childhood brain tumor (craniopharyngioma), childhood brain tumor (ependymoblastoma), childhood brain tumor (Ependymoma), childhood brain tumor (medulloblastoma), childhood brain tumor (medulloepithelioma), childhood brain tumor (pineal parenchymal tumors of intermediate differentiation), childhood brain tumor (supratentorial primitive neuroectodermal tumors and pineoblastoma), childhood brain and spinal cord tumors (other), breast cancer, breast cancer and pregnancy, childhood breast cancer, male breast cancer, childhood bronchial tumors, Burkitt lymphoma, childhood carcinoid tumor, gastrointestinal carcinoid tumor, carcinoma of unknown primary, childhood central nervous system atypical teratoid/rhabdoid tumor, childhood central nervous system embryonal tumors, primary central nervous system lymphoma, cervical cancer, childhood cervical cancer, childhood cancers, childhood chordoma, chronic lymphocytic leukemia, chronic myelogenous leukemia, chronic myeloproliferative disorders, colon cancer, childhood colorectal cancer, childhood craniopharyngioma, cutaneous T-cell lymphoma, embryonal tumors (childhood central nervous system), endometrial cancer, childhood ependymoblastoma, childhood ependymoma, esophageal cancer, childhood esophageal cancer, Ewing family of tumors, childhood extracranial germ cell tumor, extragonadal germ cell tumor, extrahepatic bile duct cancer, eye cancer (intraocular melanoma), eye cancer (retinoblastoma), gallbladder cancer, gastric (stomach) cancer, childhood gastric (stomach) cancer, gastrointestinal carcinoid tumor, gastrointestinal stromal tumor (GIST), childhood gastrointestinal stromal cell tumor, childhood germ cell tumor (extracranial), germ cell tumor (extragonadal), germ cell tumor (ovarian), gestational trophoblastic tumor, adult glioma, childhood brain stem glioma, hairy cell leukemia, head and neck cancer, adult (primary) hepatocellular (liver) cancer, childhood (primary) hepatocellular (liver) cancer, histiocytosis (Langerhans cell), adult Hodgkin lymphoma, childhood Hodgkin lymphoma, hypopharyngeal cancer, intraocular melanoma, islet cell tumors (endocrine pancreas), Kaposi sarcoma, kidney (renal cell) cancer, childhood kidney cancer, Langerhans cell histiocytosis, laryngeal cancer, childhood laryngeal cancer, adult leukemia (acute lymphoblastic), childhood leukemia (acute lymphoblastic), adult leukemia (acute myeloid), childhood leukemia (acute myeloid), leukemia (chronic lymphocytic), leukemia (chronic myelogenous), leukemia (hairy cell), lip and oral cavity cancer, adult liver cancer (primary), childhood liver cancer (primary), non-small cell lung cancer, small cell lung cancer, AIDS-related lymphoma, cutaneous T-cell lymphoma, adult Hodgkin lymphoma, childhood Hodgkin lymphoma, adult non-Hodgkin lymphoma, childhood non-Hodgkin lymphoma, primary central nervous system lymphoma, Waldenström macroglobulinemia, malignant fibrous histiocytoma of bone and osteosarcoma, childhood medulloblastoma, childhood medulloepithelioma, melanoma, intraocular (eye) melanoma, Merkel cell carcinoma, adult malignant mesothelioma, childhood mesothelioma, metastatic squamous neck cancer with occult primary, mouth cancer, childhood multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal cancer, childhood nasopharyngeal cancer, neuroblastoma, adult non-Hodgkin lymphoma, childhood non-Hodgkin lymphoma, non-small cell lung cancer, childhood oral cancer, lip and oral cavity cancer, oropharyngeal cancer, osteosarcoma and malignant fibrous histiocytoma of bone, childhood ovarian cancer, ovarian epithelial cancer, ovarian germ cell tumor, ovarian low malignant potential tumor, pancreatic cancer, childhood pancreatic cancer, pancreatic cancer (islet cell tumors), childhood papillomatosis, paranasal sinus and nasal cavity cancer, parathyroid cancer, penile cancer, pharyngeal cancer, childhood pineal parenchymal tumors of intermediate differentiation, childhood pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, childhood renal cell (kidney) cancer, renal pelvis and ureter (transitional cell cancer), respiratory tract carcinoma involving the NUT gene on chromosome 15, retinoblastoma, childhood rhabdomyosarcoma, salivary gland cancer, childhood salivary gland cancer, sarcoma (Ewing family of tumors), adult soft tissue sarcoma, childhood soft tissue sarcoma, uterine sarcoma, Sézary syndrome, skin cancer (nonmelanoma), childhood skin cancer, skin cancer (melanoma), Merkel cell skin carcinoma, small cell lung cancer, small intestine cancer, squamous cell carcinoma, squamous neck cancer with occult primary (metastatic), stomach (gastric) cancer, childhood stomach (gastric) cancer, childhood supratentorial primitive neuroectodermal tumors, cutaneous T-cell lymphoma, testicular cancer, throat cancer, thymoma and thymic carcinoma, childhood thymoma and thymic carcinoma, thyroid cancer, childhood thyroid cancer, transitional cell cancer of the renal pelvis and ureter, gestational trophoblastic tumor, adult carcinoma of unknown primary site, childhood cancer of unknown primary site, unusual cancers of childhood, transitional cell cancer of ureter and renal pelvis, urethral cancer, endometrial uterine cancer, uterine sarcoma, vaginal cancer, childhood vaginal cancer, vulvar cancer, Wilms tumor, and women's cancers.

Oocytes or Other Cells: The present invention preferably uses Xenopus laevis oocytes. Use of other cells in place of or in addition to Xenopus laevis oocytes is envisioned. These include but are not limited to human oocytes, plant protoplasts, plant parenchyma cells, plant collencyma cells, plant sclerenchyma cells, bacteria such as cyanobacteria (nostoc pruniforme), and Mare's Eggs.

Preliminary studies tested a method by the present inventor described in U.S. Pat. No. 7,135,336, which employs oocyte cytoplasm. These studies showed that the method of the '336 patent yields only low efficacy of reprogramming (less than 1%). Furthermore, these experiments were characterized by a low cell survival rate. For example, of 100,000 donor cells injected inside oocytes, approximately 80,000 cells (80%) could not withstand encapsulation and died. The methods described herein differ from the methods of the '336 patent at least by using live oocytes with intact or semi-permeabilized membranes. Without being limited to a particular mechanism, it is hypothesized that the intact or semi-permeable oocyte membrane allows only particular factors to travel across/through the membrane barrier for access to the differentiated cell. It is envisioned that the present invention may be improved by changing the size of pores in membrane.

Electroporation: The present invention preferably uses electroporation. Use of other electric, magnetic, or electromagnetic stimuli is envisioned.

### EXAMPLES

The following materials and methods were used in the examples described below.

### Cell Lines

Irradiated Mouse Embryonic Fibroblasts (iMEFs) were purchased from American R&D Systems (cat. # PSC001) and grown at 37° C. and 5% CO2 in non-pyrogenic, sterile 25 cm2, 0.2 μm ventilated cell culture flasks (T25; Corning cat. #3056, Corning, N.Y., USA) containing 5 ml of high glucose DMEM (Millipore cat. #SLM-220M) supplemented with 10% Fetal Bovine Serum (FBS; ATCC cat. # 30-2020), 1 mM sodium pyruvate (Sigma cat. #P2256), 0.1 mM non-essential amino acids (NEAA; Gibco cat. # 11140), and 1% penicillin (50 U/mL)/ streptomycin (50 μg/mL) solution (1% pen/strep; GIBCO cat. # 15140).

Human Bone Marrow Stromal Cells (BMSCs) were provided by Tulane University Center of Gene Therapy (grant from NCRR of the NIH, Grant #P40RR017447). GFP-expressing BMSCs (BMSCGFP) were stably transfected at the same facility. Prior to release, two trials of frozen, passage-1 cells were analyzed over three passages for colony forming units, cell growth, and differentiation into fat, bone, and chondrocytes.

Human Normal Foreskin Fibroblasts (BJ cells) were purchased from American Type Culture Collection (ATCC; cat. # CRL-2522). BJ cells were maintained at 37° C. and 5% CO2 in T25 culture flasks in 5 ml of Eagle's Essential Medium (ATCC cat. # 30-2003) supplemented with 10% FBS, 1 mM sodium pyruvate, 0.1 mM NEAA, and 1% pen/strep.

Human Subcutaneous Pre-adipocytes were purchased from ScienCell Research Laboratories of Carlsbad Calif., USA (cat. #7220) and cultured at 37° C. and 5% CO2 in T25 flasks coated with 0.01% poly-lysine (Sigma Cat. # P4832) and containing 5 ml of specially formulated preadipocyte medium (PAM; ScienCells cat. # 7211). PAM was supplemented with 5% FBS, 1 mM sodium pyruvate, 0.1 mM NEAA, and 1% pen/strep.

Human Peripheral Blood CD4+ T-lymphocytes (CD4TLs). Pathogen-free poietics® CD4TLs were purchased from Lonza Group, Ltd. (Lonza cat. # 2W-200, Basel, Switzerland). Originally, mature cells were isolated from normal peripheral blood using negative immunomagnetic selection directed against the CD4 surface antigen. T-Lymphocytes were maintained as a cell suspension in T25 culture flasks at 37° C. and 5% CO2 in 5 ml of lymphocyte growth medium-3 (LGM-3®, Lonza cat. # CC-3211), which was specially developed for the growth and support of human lymphocytes and dendritic cells. LGM-3® was supplemented with 10% FBS, 1 mM sodium pyruvate, 0.1 mM non-essential amino acids, 1% pen/strep, and 50 ng/ml recombinant human Interleukin-4 (R&D Systems cat. # 204-IL).

Human Buccal Mucosa Cells were obtained approximately one hour before the co-electroporation procedure. Healthy human subjects abstained from drinking coffee for one hour. The patients' mouths were rinsed twice with Listerine and, then, with sterile distilled water before swabbing. Buccal cells were collected by scrubbing a MasterAmp™ Buccal Swab Brush (Epicentre Biotechnologies cat. # MB100SP) firmly on the inside of the cheek 20 times on both sides. The brush containing cheek cells was placed into a 50 ml centrifuge tube filled with 20 ml of sterile filtered 1×PBS containing 1% pen/strep. The sample was vigorously twirled for 30 sec and, then, centrifuged at 200×g for 7 min. The pellet containing cheek cells was resuspended in 5 ml of serum-free DMEM (ATCC cat. # 30-2002) supplemented with 1 mM sodium pyruvate, 0.1 mM NEAA, and 1% pen/strep. Buccal cells were kept in a refrigerator at 4° C. before usage.

Human cervical carcinoma (HeLa) cells (routinely maintained at the Bioquark, Inc. facility) were grown at 37° C. and 5% CO2 in T25 flasks filled with 5 ml of Eagle's essential medium (ATCC cat. # 30-2003) supplemented with 10% FBS, 1 mM sodium pyruvate , 0.1 mM NEAA, and 1% pen/strep.

Human breast adenocarcinoma (MCF-7) cells were purchased from ATCC (cat. #HTB-22) and maintained in Eagle's Essential Medium supplemented with 10% FBS, 1 mM sodium pyruvate, 0.1 mM NEAA, 1% pen/strep, and 0.01 mg/ml recombinant human insulin (Eli Lilly cat. #H1-310, furnished as a gift from North-Suburban Pharmacy, Skokie, Ill.)

Preparation and Maintenance of Xenopus laevis Oocytes

All experiments were carried out in accordance with Institutional Animal Care and Use Committee (IACUC) policies. South African clawed, egg-bearing frogs (Xenopus laevis, NASCO cat. # LM00531, Fort Atkinson Wis., USA) were adopted to the new environment for two weeks at ^{˜}18° C. using a 12/12-hour light/dark cycle and were kept in carbon-filtered water supplemented with 13.3 g/gallon sodium monochloride (Rand and Kalishman, 2001). Animals were fed frog brittle (NASCO cat. # SA02764LM). Water in containers was replaced on a daily basis. Prior to surgery, frogs were anesthetized in a plastic beaker containing 1 L of 0.2% tricane solution (Sigma cat. # A5040) for up to 20 min and, then, placed on a dissecting pan filled with ice. A small incision (0.5 cm) was made through the skin layer and then the muscle layer. The bags of the ovaries were surgically removed and placed into an oocyte washing (OW) solution containing 82.5 mM NaCl (Sigma cat. #S3014), 5.0 mM HEPES (Sigma cat. #H4034), 2.5 mM KCl (Sigma cat. #P5405), 1 mM MgCl2 (Sigma cat. #M0250), 1.0 mM Na2HPO4 (Sigma cat. #S3264), and 0.5% pen/strep titrated to pH 7.4. Bags containing oocytes were disrupted with fine forceps, followed by multiple rinses in OW. After a final rinse, the remaining follicular cell layers were digested by placing material into a 0.2% collagenase type II solution (Worthington Biochemical Corporation cat # LS004176, Lakewood, N.J.) for one hour or more at room temperature. Defolliculated oocytes were rinsed in the OW solution and then placed for overnight incubation in a fresh holding buffer (HB) containing 5 mM NaCl, 5.0 mM HEPES, 2.5 mM KCl, 1 mM MgCl2, 1.0 mM Na2HPO4, 0.5% pen/strep, 1.0 mM CaCl2 (Sigma cat. #223506), 2.5 mM pyruvate, and 5% heat-inactivated horse serum (Sigma cat. # H1138) titrated to pH 7.4. Recovered oocytes in the final stage of maturity were collected in sterile 6-well cell culture clusters (Costar cat. # 3516) prefilled with an HB solution and then incubated at 17° C. in a low-temperature incubator for 24 hours before they were collected for electroporation experiments.

Co-electroporation of Xenopus laevis Oocytes with Donor Cells

Forty to fifty fresh Xenopus Laevis oocytes were placed in sterile Gene Pulser electroporation cuvettes (Bio-Rad cat. # 165-2088, Hercules, Calif.) prefilled with 400 μl of serum-free DMEM containing 1.0×105-1.5×105 cells/ml of specimen cells in suspension. Only cells with a viability above 90% were used for the experiments. Cuvettes were filled to 800 ul with serum-free DMEM and then placed into the shocking chamber. Co-electroporation of frog oocytes with the suspension of human donor cells was conducted using the following electroporation parameters: 150 v/cm/25 μF/7 pulses, with time constant at 0.5-0.7 msec. After electroporation, cuvettes containing oocytes and donor cells were placed in a low-temperature incubator at 17° C. for three hr to recover. Subsequently, donor cells were removed from the electroporation cuvette and transferred to T25 culture flasks containing iMEF feeder cells and ES-cell medium. Activated cells were left undisturbed for two days, and then the medium was refreshed.

### Culturing of Primary iPS Cells

BQ-activated donor cells were cultured on iMEF feeder cells in 0.1% gelatin-coated T25 culture flasks containing 5 ml of specially formulated Embryomax® DMEM culture medium (Millipore cat. #SLM-220-M, Danvers, Mass., USA). Medium was supplemented with 15% FBS, 1 mM sodium pyruvate, 0.1 mM NEAA, 1% pen/strep, 100 μM beta-mercaptoethanol (Gibco cat. #21985-023), and 1000 U/mL ESGRO® (Millipore cat. # ESG1106). We found that 1000 U of ESGRO® per 1.0 mL of tissue culture media is required to maintain embryonic stem (ES) cells with a stem-cell phenotype (www.millipore.com/catalogue/item/esg1106). After formation of clusters, iPS cells were separated from the feeder cells using the differential sedimentation technique previously described by Doetschman (Doetschman, 2002). Briefly, trypsinized iPS cell cultures containing iMEFs were centrifuged at 200×g, resuspended in 10 mL of complete ES culture medium, and transferred to a new T25 cell culture flask for 30 minutes at 37° C. Following incubation, the culture medium containing mostly iPS cells was transferred to a new T25 culture flask for a one-hour incubation at 37° C. to remove all remaining fibroblast feeders. Following the second incubation, the culture medium containing the iPS cells were removed, counted, and then centrifuged again at 200×g and resuspended in the ES culture medium used in our experiments. The Doetschman sedimentation method results in the removal of more than 99% of contaminating feeder cells from the iPS cell suspension.

Subculturing of Primary iPS Cells on Feeder Cells and Feeder-Free Substrates

Primary human iPS cells were separated from the feeder layer by the Doetschman differential sedimentation technique (DDST) described above and then plated on T25 culture flasks containing either iMEF feeder cells or feeder-free StemAdhere™ pluripotency substrate (Primorigen Biosciences cat. # S2070, lot # RD0507, Madison, Wis.). Subcultured iPS cell were cultured in a NutriStem™ medium purchased from StemGent (cat. # 01-0005).

### Cryopreservation of Human iPS Cells

For the cryopreservation of human iPSc-like cells, we used the standard slow-cooling freezing method. One ml of iPSc-like cells was gently resuspended in 1.5 ml cryovials (Nalgene cat. # 5011-0012, Rochester, N.Y.) containing 0.5 mL of 2×hES cell freezing medium (60% FBS, 20% hES cell culture medium, and 20% DMSO). Cryovials were transferred to 5100 Cryo 1° C. Freezing Container (Nalgene cat. # 5100-0001), refrigerated at −80° C. overnight and then rapidly transferred to liquid nitrogen.

### Alkaline Phosphatase Staining and Immunocytochemistry

An alkaline phosphatase (AP) substrate solution was prepared using Vector® Blue Alkaline Phosphatase Substrate Kit III (Vector Laboratories, Inc. cat # SK-5300, Burlingame, Calif., USA) as per the manufacturer's instructions. All immunocytochemistry studies were carried out at room temperature. All populations of iPS cells in T25 culture flasks went through the following steps: (a) the growth medium was removed, (b) washed three times with 1×PBS, c) fixed in −10° C. methanol, c) washed three times with 1×PBS, d) incubated for 20 min in 10% normal serum to suppress nonspecific binding, e) incubated for 60 min. in primary antibody diluted in 1.5% normal serum, f) washed three times with 1×PBS, g) incubated for 45 min. in the dark with secondary antibody diluted in 1.5% normal serum, h) washed three times with 1×PBS and left in 3rd rinse, i) examined under an inverted-phase contrast fluorescent microscope, j) PBS replaced with the anti-fading reagent 2% DABCO (Sigma cat # D-2522), and k) processed T25 flasks with specimens were sealed with parafilm, wrapped in aluminum foil and kept in a refrigerator at 4° C. for future references. The 2.5 μg/ml concentrations of primary and secondary antibodies and normal sera used in each staining were Oct3/4 (Santa Cruz Biotechnology Inc. cat # sc-8629, Santa Cruz, Calif.), NANOG (Santa Cruz cat # sc-30331), Sox-2 (Santa Cruz cat # sc-1720), TRA-1-60 (Santa Cruz cat # sc-21705), SSEA-1 (Santa Cruz cat # sc-21702), Rex-1 (Santa Cruz cat # sc-50669), goat-anti mouse 1 gM-TR (Santa Cruz cat # sc-2983), donkey-anti-mouse 1 gG-FITC (Santa Cruz cat # sc-2099), donkey anti-goat IgG-FITC (Santa Cruz cat # sc-2024), donkey anti-goat IgG-TR (Santa Cruz cat # sc-2783), normal donkey serum (Santa Cruz cat # sc-2044), and normal goat serum (Santa Cruz cat # sc-2043). DNA staining was performed using DAPI (Santa Cruz cat # sc-300415).

### Calculation of the Efficacy of Reprogramming of Human CD4+ Lymphocytes

We calculated the efficacy of reprogramming only human CD4+ lymphocytes. We demonstrated that, at early stages (12 h-24 h) of reprogramming, the expression of the Nanog gene in BQ-activated human CD4+ lymphocytes precedes the formation of accomplished CD4+ LiPSc-like clusters. This observation suggested the possible evaluation of the efficacy of reprogramming by scoring the number of reprogrammed cells expressing Nanog gene. This approach gave us the opportunity to calculate three to four times, with some proximity, the total number of glowing cells per specimen (GCS) in each T25 flask. Subtracting the number of nonspecific binding sites (NSB) in the control flasks from the GCS and knowing the original number of cells taken into BQ-activation (BQC), we calculated the mean and standard deviation for each treatment.

### Controls

We used the following controls:
a) ^{˜}105 donor cells were placed in an electroporation cuvette without oocytes, electroporated and incubated for 3 hr at 17° C.
b) Oocytes were placed with ^{˜}105 donor cells into the same electroporation chamber, but no electrical stimulation was applied. Cuvette containing oocytes and donor cells were incubated for 3 hr at 17° C.
c) ^{˜}105 donor cells were placed an electroporation cuvette without oocytes and and incubated for 3 hr at 17° C. before transferred to ES cell media (no electroporation applied)
d) Oocytes were electrically stimulated in electroporation cuvette in the absence of donor cells, then ^{˜}105 donor cells were transferred to 800 μl of extra-oocyte solution (electroporate) containing no oocytes and incubated for 3 hr at 17° C.
e) Oocytes were electrically stimulated in electroporation cuvette in the absence of donor cells, then ^{˜}105 of electroporated donor cells were transferred to 800 μl of electroporate containing no oocytes and incubated for 3 hr at 17° C.
f) ^{˜}105 of iMEF cells were co-electroporated with 40-50 oocytes, incubated for 3 hr at 17° C., then separated from oocytes, and transferred to T25 flask containing complete ES growth media for culturing.

### Example 1

we demonstrated that controls: “a”, “b”, “c”, and “f” to be RP-negative. In control “d” where non-electroporated donor cells were exposed for 3 hr to electroporate we detected ^{˜}0.4% RP efficacy (calculated only for CD4TLs, data not shown). In control “e” where electroporated donor cells were exposed to electroporate for 3 hr RP efficacy was elevated in comparison with control “e” and was ^{˜}0.9% (calculated only for CD4TLs, data not shown).

### Example 2

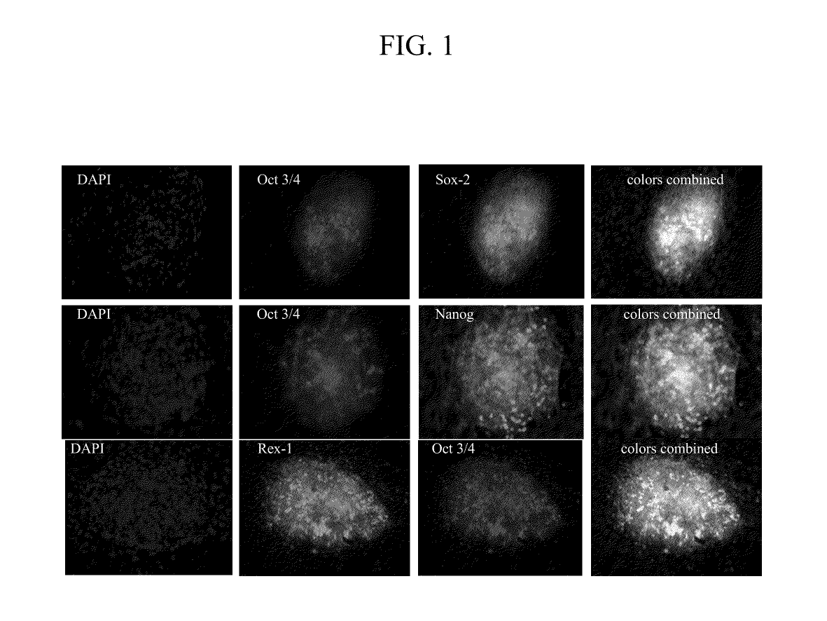

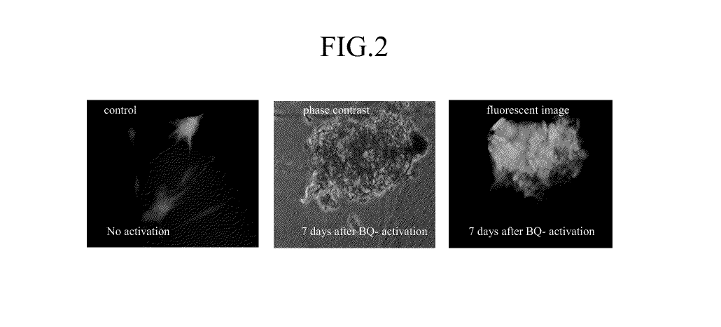

we demonstrated that BQ-activated human bone marrow stromal cells can de-differentiate into iPSc-like cells, which appeared to be indistinguishable from human embryonic stem cells in colony morphology. BMSCs strongly expressed the pluripotency-associated transcription factors Oct3/4, SOX-2, Nanog and Rex-1 (FIG. 1). In separate studies, we used BMSC-GFP to show a direct link between activated donor cells and cells that form iPSc-like clusters (FIG. 2).

### Example 3

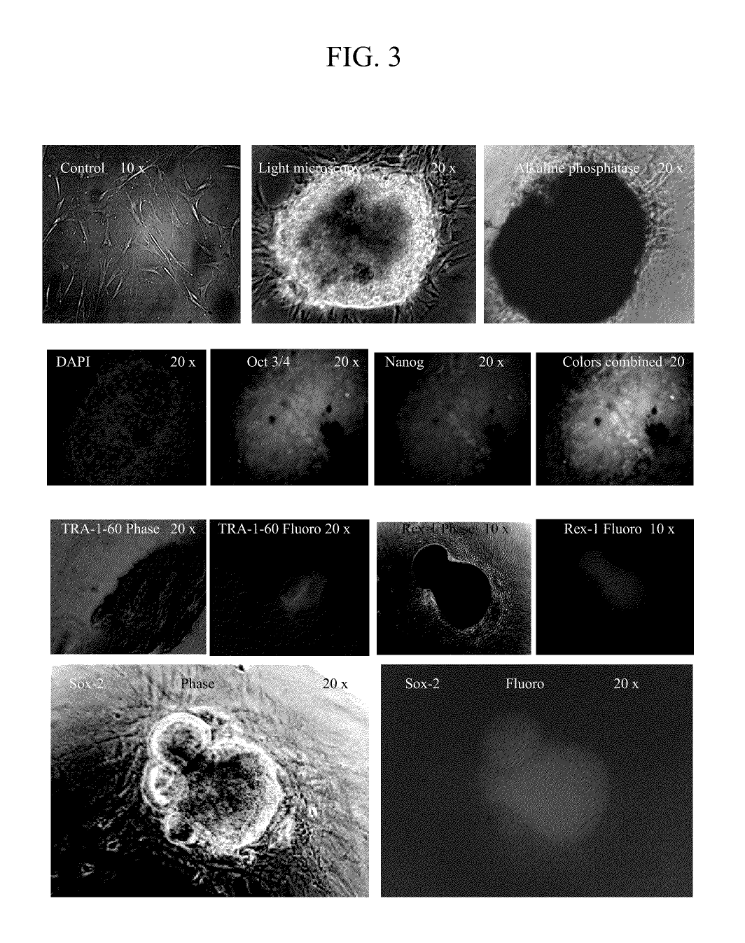

BQ-activated BJ cells de-differentiated into iPSc-like cells, which exhibited strong alkaline phosphatase activity and resembled human embryonic stem cells in both their colony morphology and the expression of major stem cell markers, such as Oct3/4, Nanog, SOX-2, TRA-1-60 and Rex-1 (FIG. 3).

### Example 4

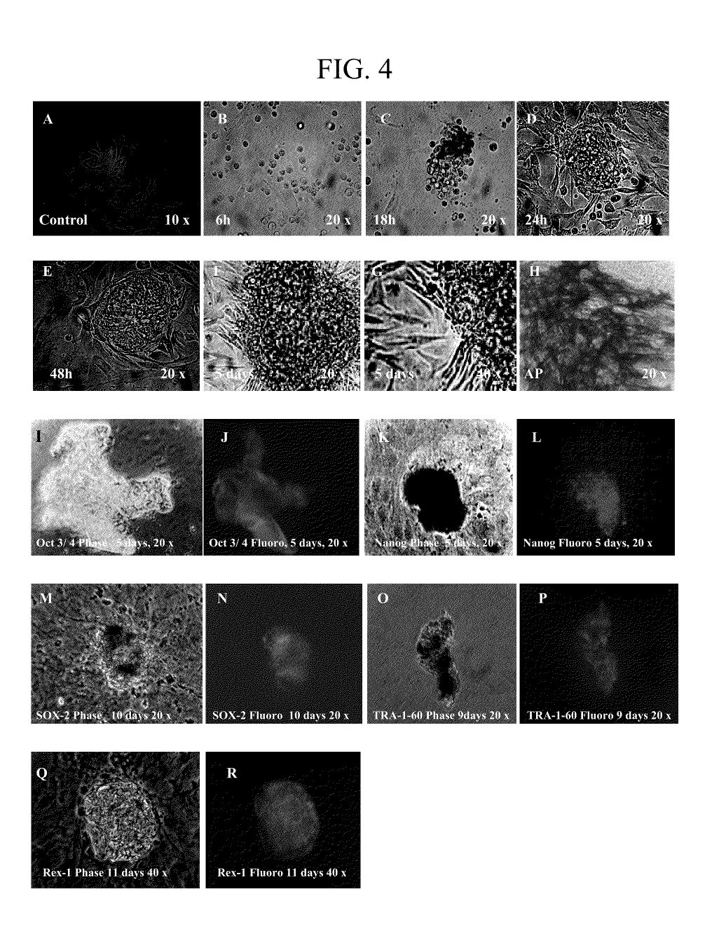

co-electroporated HPA cells de-differentiated into human iPSc-like cells, which appeared to be indistinguishable from human embryonic stem cells in colony morphology. HPA-derived iPSc-like cells displayed strong alkaline phosphatase activity. The pluripotency-associated transcription factors Oct3/4, Nanog, SOX-2, TRA-1-60 and Rex-1 were strongly expressed in these developing HPA iPSc-like colonies (FIG. 4).

### Example 5

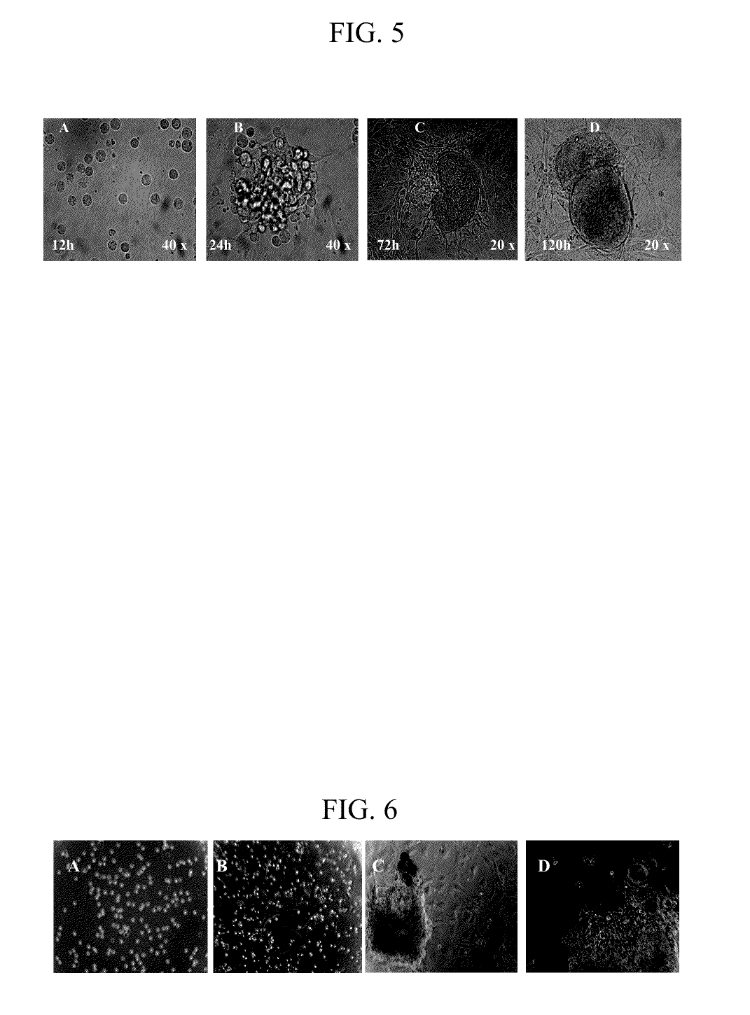

we demonstrated that, by the fourth day of subculturing, primary HPA-iPSc-like cells readily form secondary iPSc-like clusters. Microphotographs on (FIG. 5B, 5C, 5D) show different stages of formation in the same secondary HPA-iPSc-like cluster. FIG. 5 A is a randomly chosen observation area not relevant to the clusters depicted on FIG. 5B, 5C, 5D). We did not examine the immunostaining profiles for these newly obtained HPA-iPSc-like clusters because, in this particular experiment, our task was to demonstrate that primary HPA-iPSc-like cells can survive deep freezing and subsequently be subcultured and produce viable (growing) iPSc-like clusters. We demonstrated that this survival is possible.

### Example 6

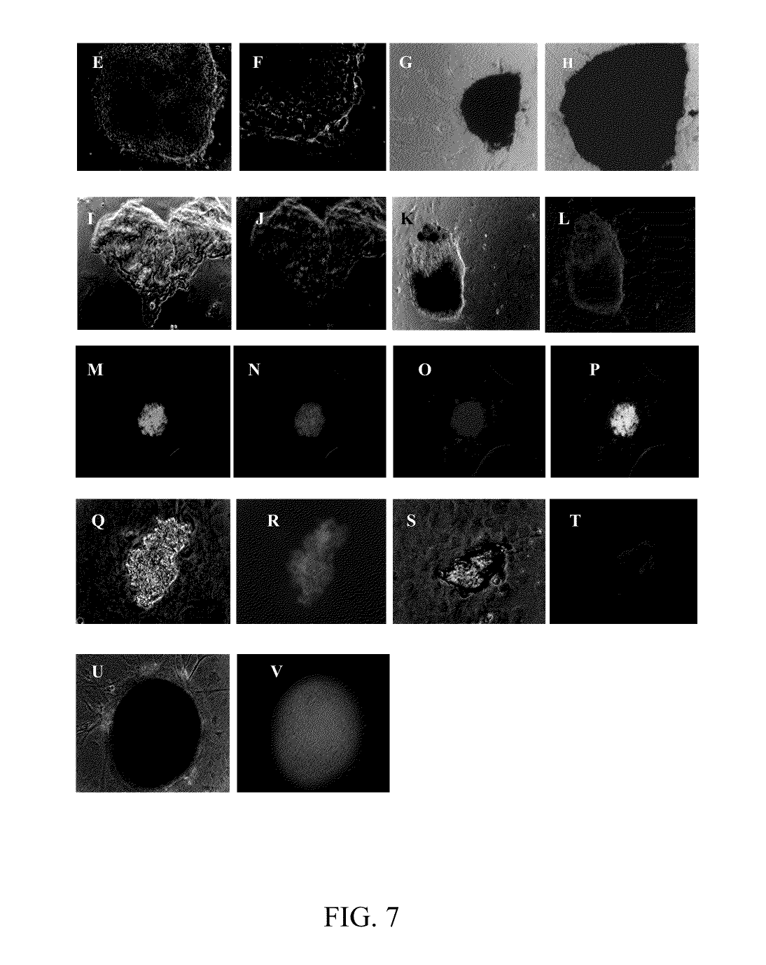

we demonstrated that BQ-activated human CD4+ T-lymphocytes, when transferred directly to feeder cells, rapidly (on the third to fifth day) form iPSc-like colonies. Our results unambiguously indicate that human CD4+ T-lymphocytes de-differentiated into iPSc-like cells. They also displayed high alkaline phosphatase activity. Expression of major stem cell markers, such as Oct3/4, Nanog, SOX-2, TRA-1-60 and Rex-1 were also strongly expressed in these developing human CD4+ L-iPSc-like cell colonies (FIG. 6A-FIG. 6X).

### Example 7

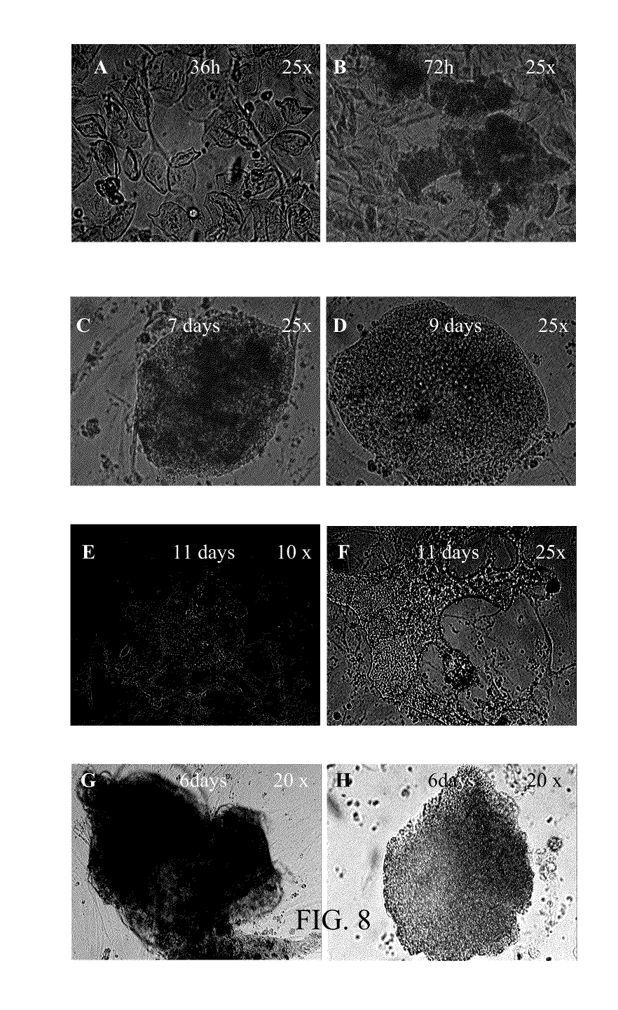

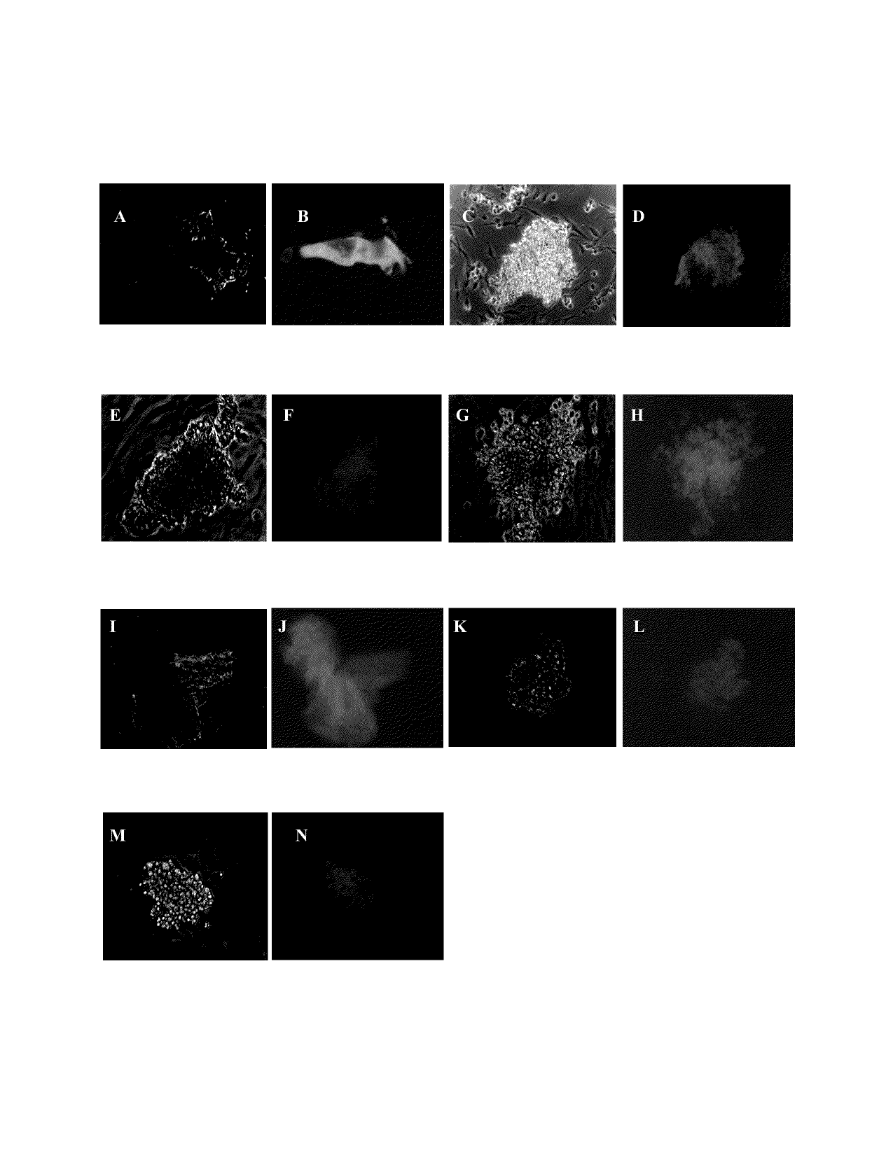

we demonstrated that isolated human buccal mucosa cells, when exposed to electroporation in the presence of living Xenopus laevis oocytes, can de-differentiate into iPSc-like cells, which appeared to be indistinguishable from human embryonic stem cells in colony morphology and the expression of pluripotency-associated transcription factors. Human buccal cell-derived iPS-c-like cells (BU-iPSc) revealed high levels of expression of the pluripotency-associated transcription factors Oct3/4, Nanog, SOX-2, TRA-1-60 and Rex-1. (FIG. 7-FIG. 8).

### Example 8

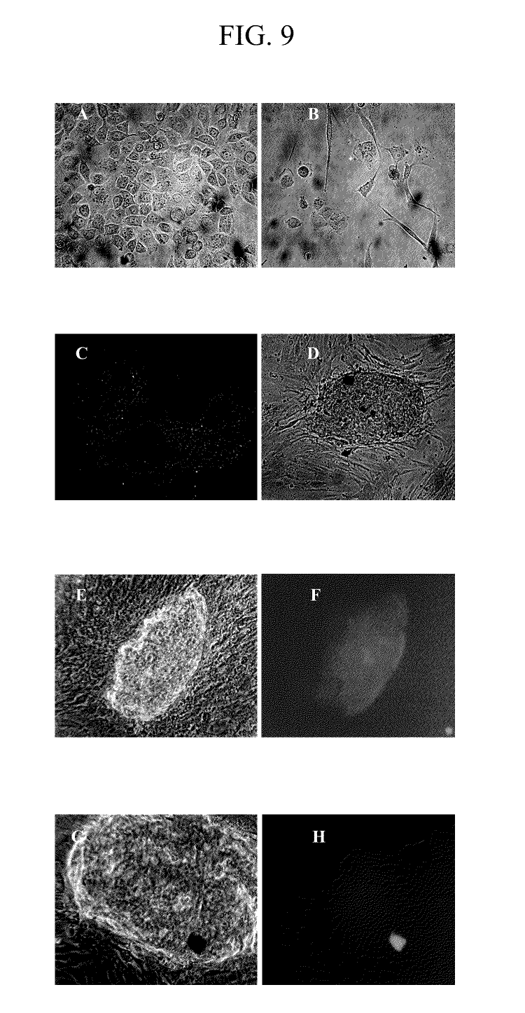

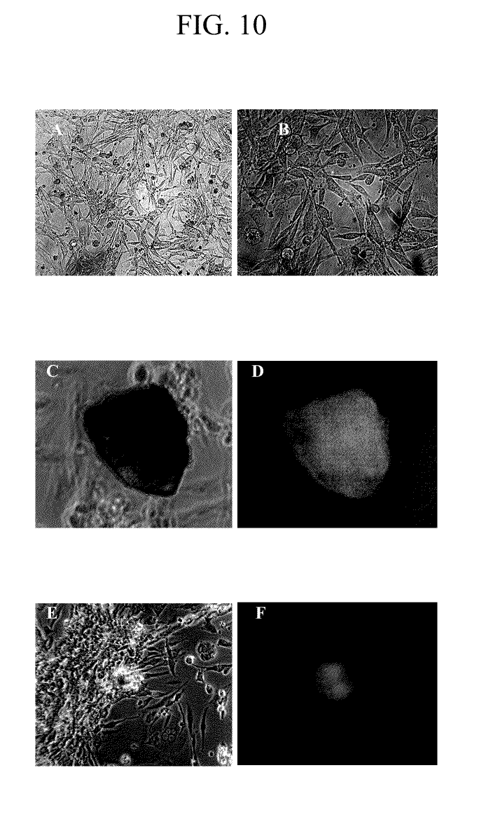

in this set of experiments was designed to investigate if selected cancer cell lines (HeLa and MCF-7) are RP-responsive to BQ-activation. We demonstrated that human cervical carcinoma and breast adenocarcinoma cells both de-differentiate and partially de-differentiate into iPSc-like clusters positively expressing the Oct 3/4 and Nanog genes (FIG. 9-FIG. 10).

### Example 9

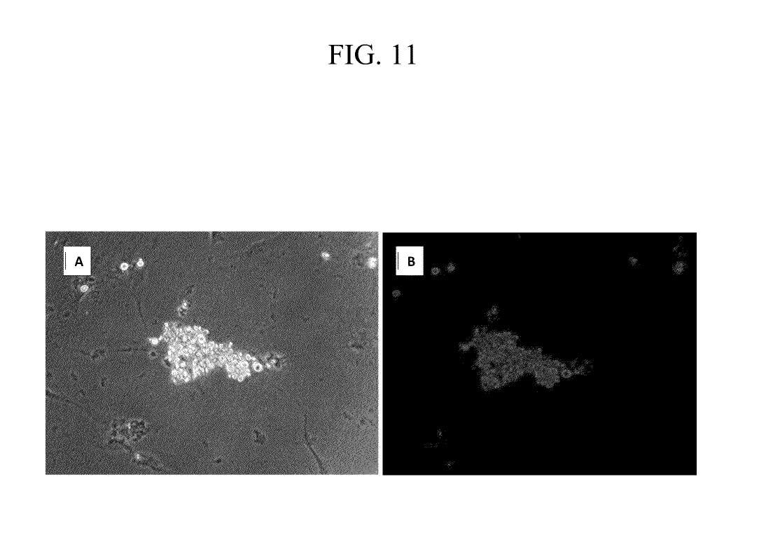

In this set of experiments we calculated the efficacy of reprogramming of human CD4+ T-Lymphocytes. Shortly after BQ activation (12 h-24 h), CD4+ lymphocytes start to express the Nanog gene. By that time, single activated cells, as well as developing iPSc-like clusters can be clearly observed (FIG. 11A-FIG. 11B). This observation suggested the possible evaluation of the efficacy of reprogramming by scoring the number of reprogrammed cells expressing Nanog gene. This approach gave us the opportunity to calculate three to four times, with some proximity, the total number of glowing cells per specimen (GCS) in each T25 flask. Subtracting the number of nonspecific binding sites (NSB) in the control flasks from the GCS and knowing the original number of cells taken into BQ-activation (BQC), we calculated the mean and standard deviation for each treatment. The findings on the efficacy of reprogramming of human CD4TL are present in TABLE 1.

TABLE 1

| Cells | BQC | NSB | GCS-1 | GCS-2 | GCS-3 | GCS-4 | Mean RP % ± SD |
|---|---|---|---|---|---|---|---|
| CD4 + L | 10⁵ | 201 ± 15 | 25,321 | 22,256 | 27,355 | 19,285 | 23.4 ± 3.5 |

### REFERENCES

1. Atkinsona D. L., Stevensona T. J., Parkbe I. J., Riedyc m. D., Milashd B., Odelberga S. J., 2006.Cellular Electroporation Induces Dedifferentiation in Intact Newt Limbs. Dev Biol. 1; 299 (1): 257-271
2. Buono R. J., Linser P. J., 1992. Transgenic Zebrafish by Electroporation. Bio-Rad US/EG Bullet. 1354, September 9. pp. 1-4
3. Bussard K. M., Boulanger C. A., Booth B. W., Bruno R. D., Smith G. H., 2010. Reprogramming Human Cancer Cells in the Mouse Mammary Gland. Cancer Res; 70 (15): 6336-43.
4. Cho H. J., Lee C. S., Kwon Y. W., Paek J. S., Lee S. H., Hur J., Lee I. J., Roh T. Y., Chu I. S., Leem S. H., Kim Y, Kang H. J., Park
5. Y. B., Kim H. S., 2010. Induction of pluripotent stem cells from adult somatic cells by protein-based reprogramming without genetic manipulation. Blood, 116 (3): 386-395
6. Condic M. L., Rao M., 2008. Regulatory Issues For Personalized Pluripotent Cells. Stem Cells. Vov. 26 (11): 2753-2758.
7. Doetschman T., 2002. Gene Targeting in Embryonic Stem Cells. A Lab. Handbook. Acad. Press; San Diego.
8. Falk J., Drinjakovic I., Leung K. M., Dwivedy A., Regan A. G., Piper M., Holt C. E., 2007. Electroporation of cDNA/Morpholinos to targeted areas of embryonic CNS in Xenopus. BMC Dev Biol. 7: 107.
9. Frenster J. H., Hovsepian J. A., 2007. Models of Embryonic Gene-Induced Initiation and Reversion of Adult Neoplasms. AACR-NCI-EORTC International Conference: “Molecular Targets and Cancer Therapeutics: Discovery, Biology, and Clinical Applications”, p. 258-9, October 25. San-Francisco, Calif.
10. Gonda K., Fowler J., Katoku-Kikyo N., Haroldson J., Wudel J., Kikyo N., 2003. Reversible disassembly of somatic nucleoli by the germ cell proteins FRGY2a and FRGY2b. Nat Cell Biol., March; 5 (3):205-10
11. Granneman J. G., Li P., Lu Y., Tilak J., 2004. Seeing the trees in the forest: selective electroporation of adipocytes within adipose tissue. Am. J. Physiol. Endocrinol. Metab. 287: E574-E582.
12. Hakelien A-M., Landsverk H. B, Robl J. M., Skalhegg B. S., Collas P., 2002. Reprogramming fibroblasts to express T-cell functions using cell extracts. Nature Biotechnology 20, 460-466.
13. Hansis C., Barreto G., Maltry N., Niehrs C., 2004. Nuclear reprogramming of human somatic cells by xenopus egg extract requires BRG1. Curr Biol. August 24; 14 (16):1475-80
14. Hockemeyer D., Soldner F., Beard C., Gao Q., Mitalipova M., DeKelver R. C., Katibah G. E., Amora R., Boydston E. A., Zeitler B., Meng X., Miller J. C., Zhang L., Rebar E. J., Gregory P. D., Urnov F. D., Jaenisch R., 2009. Efficient targeting of expressed and silent genes in human ESCs and iPSCs using zinc-finger Nucleases. Nat. Biotech. 13 August, pp. 1-9
15. Hong H., Takahashi K., Ichisaka T., Aoi T., Kanagawa O., Nakagawa M., Okita K., Yamanaka, S., 2009. Suppression of induced pluripotent stem cell generation by the p53-p21 pathway. Nature 460, 1132-1135.
16. Hostetler H. A., Peck S. L., Muir W. M., 2003. High efficiency production of germ-line transgenic Japanese medaka (Oryzias latipes) by electroporation with direct current-shifted radio frequency pulses. Transgenic Research 12: 413-424.
17. Huangfu D., Osafune K., Maehr R., Guo W., Eijkelenboom A., Chen S., Muhlestein W., Melton D. A., 2008. Induction of pluripotent stem cells from primary human fibroblasts with only Oct4 and Sox2. Nature Biotech. 26, 1269-1275.
18. Jee M. K., Kim J. H., Han Y. M., Jung S. J., Kang K. S., Kim D. W., Kang S. K., 2010. DHP-derivative and low oxygen tension effectively induces human adipose stromal cell reprogramming. PLoS One. 2010 Feb. 9; 5 (2):e9026.
19. Judson R. L., Babiarz J. I., Venere M., Blelloch R., 2009. Embryonic stem cell-specific microRNAs promote induced Pluripotency. Nature Biotech. volume 27 number 5, p.p. 459-461.
20. Katoh M., Chen G., Roberge E., Shaulsky G., Kuspa A., 2007. Developmental Commitment in Dictyostelium Discoideum. Eukariotic Cell Nov., p. 2038-2045 Vol. 6, No. 11.
21. Kikyo N., Wade P. A., Guschin D., Ge H., Wolffe A. P., 2000. Active remodeling of somatic nuclei in egg cytoplasm by the nucleosomal ATPase ISWI. Science. September 29; 289 (5488):2360-2.
22. Kim K., Doi A., Wen B., Ng K., Zhao R., Cahan P., Kim J., Aryee M. J., Ji H., Ehrlich L. I., Yabuuchi A., Takeuchi A., Cunniff K. C., Hongguang H., McKinney-Freeman S., Naveiras O., Yoon T. J., Irizarry R. A., Jung N., Seita J., Hanna J., Murakami P., Jaenisch R., Weissleder R., Orkin S. H., Weissman I. L., Feinberg A. P., Daley G. Q., 2010. Epigenetic memory in induced pluripotent stem cells. Nature. July 19. [Epub ahead of print].
23. Kim Y. D., Kang S. M., Min J. Y., Choi W. K., Jeong M. J., Karigar C. S., Choi M. S., 2010. Production of tropane alkaloids during de-differentiation of Scopolia parviflora calli. J Nat Prod. February 26; 73 (2):147-50.
24. Kragl M., Knapp D., Nacu E., Khattak S., Maden M., Epperlein H. H., Tanaka E. M., 2009. Cells keep a memory of their tissue origin during axolotl limb regeneration. Nature 460, 60-65.
25. Maria O. M., Khosravi R. , Mezey E., Tran S. D., 2007. Cells from bone marrow that evolve into oral tissues and their clinical applications. Oral Diseases, 13: 11-16
26. Markoulaki S., Hanna J., Beard C., Carey B. W., Cheng A. W., Lengner C. J., Dausman J. A., Fu D., Gao Q., Wu S., Cassady J. P., Jaenisch R., 2009. Transgenic mice with defined combinations of drug-inducible reprogramming factors. Nature Biotechnology, 27: 169-171.
27. Meissner A., Wernig M., Jaenisch R., 2007. Direct reprogramming of genetically unmodified fibroblasts into pluripotent stem cells. Nature Biotechnology 25: 1177-1181.
28. Miyamoto K., Furusawa T., Ohnuki M., Goel S., Tokunaga T., Minami N., Yamada M., Ohsumi K., Imai H., 2007. Reprogramming events of mammalian somatic cells induced by Xenopus laevis egg extracts. Mol Reprod Dev. October; 74 (10):1268-77.
29. Murata K., Kouzarides T., Bannister A. J., Gurdon J. B., 2010. Histone H3 lysine 4 methylation is associated with the transcriptional reprogramming efficiency of somatic nuclei by oocytes. Epigenetics Chromatin. February 4; 3 (1):4.
30. Rand, Kalishman J., 2001. Xenopus Care, Health & Disease: A Brief Overview. Sareen D., Svendsen C. N., 2010. Stem cell biologists sure play a mean pinball. Nature Biotechnology, Volume: 28, pp: 333-335.
31. Tamada H., Van Thuan N., Reed P., Nelson D., Katoku-Kikyo N., Wudel J., Wakayama T., Kikyo N., 2007. Chromatin decondensation and nuclear reprogramming by nucleoplasmin. Mol Cell Biol. September; 27 (18):6580.
32. Travaglini L., Vian L., Billi M., Grignani F., Nervi C., 2008. Epigenetic reprogramming of breast cancer cells by valproic acid occurs regardless of estrogen receptor status. Int J Biochem Cell Biol. 2009 January; 41 (1):225-34. Epub, August 22.
33. Straube W., Tanaka E. M., 2006. Reversibility of the Differentiated State: Regeneration in Amphibians Artificial Organs 30 (10):743-755, Blackwell Publishing, Inc.
34. Wernig M., Lengner C. J., HanFna J., Lodato M. A., Steine E., Foreman R., Staerk J., Markoulaki S., Jaenisch R., 2008. A drug-inducible transgenic system for direct reprogramming of multiple somatic cell types Nat. Biotechnol. August; 26 (8): 916-924.
35. Yoshida Y., Takahashi K., Okita K., Ichisaka T., Yamanaka S., 2009. Hypoxia Enhances the Generation of Induced Pluripotent Stem Cells. Cell Stem Cell, 27 Aug. 2009 pp. 1-5
36. Zhao J., Morozova N., Williams L., Libs L., Avivi Y., Grafi G., 2001. Two Phases of Chromatin Decondensation During Dedifferentiation of Plant Cells: Distinction Between Competence for Cell-Fate Switch and a Commitment for S Phase. The J. of Biol. Chem., Mar. 26, 2001.
37. Zhou H., et al., 2009. Generation of Induced Pluripotent Stem Cells Using Recombinant Proteins. Cell Stem Cell, 4, 381- 384.

I claim:

1. A method of generating an induced pluripotent stem cell from a differentiated cell comprising co-electroporating the differentiated cell with a live oocyte.

2. The method of claim 1 wherein the live oocyte is derived from Xenopus laevis.

3. The method of claim 1 wherein the differentiated cell is selected from the group consisting of a fibroblast, a preadipocyte, a lymphocyte, a buccal cell, and a cancer cell.

4. The method of claim 1 wherein the induced pluripotent stem cell expresses a gene product selected from the group consisting of Oct-3/4, NANOG, SOX2, TRA-1-60, and Rex-1.

5. The method of claim 1 wherein the co-electroporating comprises stimulating the differentiated cell with seven 50-volt/25 nF/ impulses with 1-second intervals and with a time constant equal to 0.5-0.7 milliseconds.

6. A method of generating an induced pluripotent stem cell from a differentiated cell comprising electroporating the differentiated cell.

7. The method of claim 6 wherein the differentiated cell is selected from the group consisting of a fibroblast, a preadipocyte, a lymphocyte, a cord blood cell, a cancer cell, and a buccal cell.

8. The method of claim 6 wherein the induced pluripotent stem cell expresses a gene product selected from the group consisting of Oct-3/4, NANOG, SOX2, TRA-1-60, and Rex-1.

9. The method of claim 6 wherein the electroporating comprises stimulating the differentiated cell with seven 50-volt/25-nF impulses with 1-second intervals and with a time constant equal to 0.5-0.7 milliseconds.

10. A method of generating an induced pluripotent stem cell from a differentiated cell comprising co-incubating the differentiated cell with a live oocyte.

11. The method of claim 10 wherein the live oocyte is derived from Xenopus laevis.

12. The method of claim 10 wherein the differentiated cell is selected from the group consisting of a fibroblast, a preadipocyte, a lymphocyte, a cord blood cell, a cancer cell, and a buccal cell.

13. The method of claim 10 wherein the induced pluripotent stem cell expresses a gene product selected from the group consisting of Oct-3/4, NANOG, SOX2, TRA-1-60, and Rex-1.

14. The method of claim 10 wherein the differentiated cell is co-incubated with the live oocyte for about 3 hours.

15. An induced pluripotent stem cell line generated by the method of claim 1, claim 6, or claim 10.
